(19) United States
(12) Patent Application Publication
(10) Pub. No.: US 2007/0185141 A1
(43) Pub. Date: Aug. 9, 2007

(54) Thiamine composition for enhancement of physical and mental energy, and related method

(76) Inventor: Tim Patterson, Colorado Springs, CO (US)

Correspondence Address: Stephen T. Sullivan, 7001 E. Pardise Drive, Scottsdale, AZ, 85254, US

(21) Appl. No.: 11/353,298

(22) Filed: Feb. 9, 2006

(51) Int. Cl. A61K 31/522; C07D 473/26

(52) U.S. Cl. 514/263.3; 514/276; 544/274; 544/327

(57) ABSTRACT

A composition that includes thiamine disulfide diisobutyrate and caffeine. Related methods also are provided.

### BACKGROUND OF THE INVENTION

1. Field of the Invention

The present invention relates to biochemistry and nutraceuticals or dietary supplements. More specifically, it relates to compositions and methods for enhancing mental and physical energy and mental focus.

2. Description of the Related Art

The importance of mental and physical well being of course cannot be overstated. There is and has been a continual search for compositions and methods that enhance mental and physical energy, mental focus, and the like. This class of drugs and nutraceuticals, which has come to be referred to as “nootropics,” is of growing importance and public awareness.

A number of approaches have been reported for enhancing physical and mental energy and focus. One such approach as reported in the literature involves the use of certain thiamine derivatives, such as sulbutiamine (isobutyryl-thiamine disulfide), for enhancing neurological function. In Tiev et al., “Treatment of Chronic Postinfectionous Fatigue: Randomized Double-Blind Study of Two Doses of Sulbutiamine (400-600 mg/day) Versus Placebo,” Rev. Med. Interne., October 1999, Vol. 20, No. 10, p. 912-918, the authors report favorable results with using sulbutiamine in treating chronic postinfectious fatigue. Possible benefits of sulbutiamine also were reported for treatment of asthenia (loss of strength or energy) in Sha SN, “Adjuvant Role of Vitamin B. Analogue (Sulbutiamine) With Anti-Infective Treatment In Infection Associated Asthenia,” J. Assoc. Physicians India, September 2003, Vol. 51, p. 891-895. The use of sulbutiamine, however, is not reported as having pronounced efficacy in enhancing physical and mental energy and mental focus.

### OBJECTS OF THE INVENTION

Accordingly, an object of the present invention is to provide compositions and methods that enhance mental and/or physical energy.

Another object of the invention is to provide compositions and methods that enhance mental focus.

Another object of the invention is to provide compositions and methods that make thiamine derivatives such as sulbutiamine efficacious or enhance their effectiveness for improving energy and/or mental focus.

Additional objects and advantages of the invention will be set forth in the description which follows, and in part will be apparent from the description, or may be learned by practice of the invention. The objects and advantages of the invention may be realized and obtained by means of the instrumentalities and combinations pointed out in the appended claims.

### SUMMARY OF THE INVENTION

To achieve the foregoing objects, and in accordance with the purposes of the invention as embodied and broadly described in this document, a composition is provided that comprises thiamine disulfide diisobutyrate and caffeine. The caffeine preferably but optionally is present in the composition in an amount of at least about 50 milligrams per daily dose. More preferably it is present in an amount of about 50 milligrams per daily dose to about 250 milligrams per daily dose. In presently preferred embodiments and methods, caffeine dosages of about 125 milligrams per daily dose or 250 milligrams per daily dose are preferred.

Again, preferably but optionally, the thiamine disulfide diisobutyrate is present in an amount of at least about 200 milligrams per daily dose, and more preferably about 200 milligrams per daily dose to about 400 milligrams per daily dose. In some applications the preferred dosage is about 200 milligrams per daily dose, and others, for example, about 400 milligrams per daily dose.

The composition also may comprise an excipient, and/or a microemulsifying agent for microemulsifying the thiamine disulfide diisobutyrate and the caffeine.

The composition preferably comprises about 2.5 to 10 parts thiamine disulfide diisobutyrate for each part of the caffeine. The ratio may comprise about 2.5 parts thiamine disulfide diisobutyrate for each part of the caffeine in some applications and, in others, for example, it may comprise about 10 parts thiamine disulfide diisobutyrate for each part of the caffeine.

The composition may be or comprise a solid, it may be located in or packaged as a capsule, it may be in tablet form, and the like. In presently preferred embodiments, it may be in a microemulsified form.

In accordance with another aspect of the invention, a composition is provided that comprises thiamine disulfide diisobutyrate and at least one of an adenosine antagonist, a cyclic AMP enhancer, a phosphodiesterase inhibitor, and an epinephrine release stimulant. The composition may comprise thiamine disulfide diisobutyrate, and all or any combination of the adenosine antagonist, the cyclic AMP enhancer, the phosphodiesterase inhibitor, and the epinephrine release stimulant. It may comprise ephedrine. The adenosine antagonist, the phosphodiesterase inhibitor and/or the epinephrine release stimulant may comprise caffeine. The cyclic AMP enhancer may comprise at least one of forskohlin and sclareline. The phosphodiesterase inhibitor also may comprise at least one methyl zanthene. The epinephrine release stimulant also may comprise Ridlin.

In accordance with another aspect of the invention, a method is provided for enhancing mental acuity and physical energy in a subject. The method comprises administering to the subject a composition comprising thiamine disulfide diisobutyrate and caffeine. The method preferably comprises combining the thiamine disulfide diisobutyrate and caffeine as described herein above.

In accordance with still another aspect of the invention, a method is provided for enhancing mental acuity and physical energy in a subject. The method comprises administering to the subject a composition comprising thiamine disulfide diisobutyrate and at least one of an adenosine antagonist, a cyclic AMP enhancer, a phosphodiesterase, and an epinephrine release stimulant. In accordance with still another aspect of the invention, a method for making a composition that comprises thiamine disulfide diisobutyrate, caffeine and an excipient. The method comprises: (a) bringing the excipient to about 45° C.; (b) after completing (a), mixing the thiamine disulfide diisobutyrate with the excipient at about 45° C. until a thiamine disulfide diisobutyrate solution is obtained;

(c) after completing (b), mixing the caffeine with the thiamine disulfide diisobutyrate solution at about 45° C. until a composition solution is obtained.

The method preferably but optionally comprises bringing the composition solution to about 40° C.; and filling a capsule with the composition solution at about 40° C.

### DETAILED DESCRIPTION OF THE PREFERRED EMBODIMENTS AND METHODS

Reference will now be made in detail to the presently preferred embodiments and methods of the invention as illustrated in the accompanying drawings, in which like reference characters designate like or corresponding parts throughout the drawings. It should be noted, however, that the invention in its broader aspects is not limited to the specific details, representative devices and methods, and illustrative examples shown and described in this section in connection with the preferred embodiments and methods. The invention according to its various aspects is particularly pointed out and distinctly claimed in the attached claims read in view of this specification, and appropriate equivalents.

In accordance with one aspect of the invention, a composition is provided that comprises thiamine disulfide diisobutyrate and caffeine. Caffeine is a known stimulant. It increases nor-epinephrine secretion and enhances neural activity in a number of areas of the brain. Caffeine is reportedly a competitive antagonist at adenosine receptors. Adenosine, a neucloside, is an important neuro-modulator of a number of central nervous system functions. Adenosine normally produces a mild sedative effect when it activates certain adenosine receptor sites. Caffeine is believed to interfere with this activation.

The caffeine preferably but optionally is present in the composition in an amount of at least about 50 milligrams per daily dose. More preferably it is present in an amount of about 50 milligrams per daily dose to about 250 milligrams per daily dose. When administered in the form of tablets or capsules, for example, containing about 1.5 to 1.6 grams per tablet, dosages of about 125 milligrams per tablet are preferred, and a preferred dosage would be one to two tablets or capsules daily.

The thiamine disulfide diisobutyrate preferably but optionally is present in an amount of at least about 200 milligrams per daily dose, and more preferably about 200 milligrams per daily dose to about 400 milligrams per daily dose. Using the same illustrative example as about, for a 1.5 to 1.6 gram tablet or capsule, the preferred dosage is about 200 milligrams per tablet or capsule, with a recommended daily dosage of one to two tablets or capsules per day.

The composition preferably comprises about 2.5 to 10 parts thiamine disulfide diisobutyrate for each part of the caffeine. The ratio may comprise about 2.5 parts thiamine disulfide diisobutyrate for each part of the caffeine in some applications and, in others, for example, it may comprise about 10 parts thiamine disulfide diisobutyrate for each part of the caffeine.

Preferred forms of this composition comprise one or more excipients. The particular excipient or excipients used normally will depend upon the specifics of the intended application. Presently preferred excipients include CAPRYOL® PGMC and CAPRYOL® 90, both commercially available from Gattefosse, S. A. of Saint-Priest Cedex, France (“Gattefosse”). Another presently preferred excipient is Gelucire® 44/14, commercially available from Gotfosse of France. Gelucire® 44/14 is a mixture of glycerol and PEG1500 esters of long fatty acids. The “44” designation identifies the melting point and the “14” identifies its hydrophilic/lipophilic balance (“HLB”).

The composition preferably is or comprises a solid, including but not limited to a solid, a solid-liquid suspension, a solid-gel suspension, and the like. It may be prepared as a tablet, capsule, gelcap, microemulsion, and/or other or like forms. Microemulsified forms and methods for microemulsifying the composition are described in assignee Biotest Laboratories, LLC's U.S. patent application Ser. No. 10/910,548, the entire specification of which is hereby incorporated by reference as if fully set forth herein.

In accordance with another aspect of the invention, a method is provided for making a composition comprising thiamine disulfide diisobutyrate, caffeine and an excipient. The method comprises:

(a) bringing the excipient to about 45° C.;

(b) after completing (a), mixing the thiamine disulfide diisobutyrate with the excipient at about 45° C. until a thiamine disulfide diisobutyrate solution is obtained;

(c) after completing (b), mixing the caffeine with the thiamine disulfide diisobutyrate solution at about 45° C. until a composition solution is obtained. When mixing in the thiamine disulfide diisobutyrate and caffeine, each component should be blended to achieve a solution prior to mixing in the next ingredient. This method preferably but optionally comprises bringing the composition solution to about 40° C., and filling a capsule with the composition solution at about 40° C.

An illustrative example of a preferred implementation for this method will now be described. It will be understood, however, that this illustrative example is provided to facilitate an understanding of this aspect of the invention, and not by way of limitation.

### EXAMPLE

Excipients Capryol PGMC® and Gellucire® 44/14 were added to a mixing vessel. The Capryol PGMC® constituted 60% of the combined excipient mass and the Gellucire® 44/14 constituted 40% of the combined excipient mass. This combined excipient mass is assumed to constitute about 300 to 325 parts of a total finished product of about 650 parts. Heat was applied to the vessel as the excipient components were stirred until the excipient mixture reached a temperature of about 45° C. and these two components were well mixed and integrated. At that point, about 200 parts of thiamine disulfide diisobutyrate were added with the excipients in the vessel and the combined mixture was heated and stirred. The heating was used to keep the mixture at a temperature of about 45° C., and mixing continued until the components formed a solution or substantially heterogeneous suspension. In addition, the following ingredients were added:

Microcrystalline Cellulose . . . 33.566%

Croscarmelose . . . 19.580%

Calcium Carbonate . . . 13.986%

Sorbitol . . . 8.392%

Stearic Acid(vegetable) . . . 1.119%

Magnesium Stearate(vegetable) . . . 0.671%

The resulting solution (or suspension) is referred to herein as a thiamine disulfide diisobutyrate solution. At this point, about 125 parts of caffeine were added to the thiamine disulfide diisobutyrate solution and stirred at a temperature of about 45° C. until a solution or substantially heterogeneous suspension is obtained. This solution is referred to herein as the “composition solution.” The composition solution was then cooled to about 40° C., and was filled into 00 capsules with the composition solution at about 40° C. The active ingredients were combined with sufficient excipient to fill the capsules to the maximum practical amount. In this example, each capsule contains about 325 mg of the active ingredients, i.e., 200 mg of thiamine disulfide diisobutyrate and 125 mg of caffeine.

In accordance with another aspect of the invention, a method is providing for enhancing mental acuity and physical energy in a subject. The method comprises administering to the subject a composition comprising thiamine disulfide diisobutyrate and caffeine. Compositions suitable for the practice of this aspect of the invention include those disclosed above.

In accordance with another aspect of the invention, a composition is provided that comprises thiamine disulfide diisobutyrate and at least one of an adenosine antagonist, a phosphodiesterase, an epinephrine release stimulant, and a cyclic AMP enhancer. Compositions within the scope of this aspect of the invention comprise various combinations and mixtures of these components, or all of them.

As noted herein above, adenosine is an important neuro-modulator of a number of central nervous system functions. Adenosine activates certain adenosine receptor sites, such as G protein-linked receptors, to produce its effects. Adenosine antagonists interfere with this mechanism, for example, by blocking these receptor sites. Adenosine antagonists according to this aspect of the invention therefore may comprise any ingredient or combination of ingredients that limit the concentration of adenosine and/or impair or impede its mechanisms. Caffeine is an example of such adenosine antagonists.

The inclusion of one or more cyclic AMP enhancers also influences metabolic and other physiological effects. The activation of a G protein-linked receptor by neurotransmitters cause the production of cyclic AMP. Cyclic AMP is a “second messenger” that regulates ion channels in cell membranes, actives kinase enzymes which in turn initiate enzymatic reactions, etc. in response to G protein-linked receptor activation. In this aspect of the invention, a cyclic AMP enhancer is provided to enhance production of cyclic AMP and thus to enhance these effects. The cyclic AMP enhancer may comprise any substance or ingredient that has the effect of enhancing cyclic AMP production or function. Examples of such cyclic AMP enhancers include forskohlin and sclareline.

The phosphodiesterase inhibitor similarly may be or comprise any ingredient or substance that inhibits phosphodiesterase. The function of phosphodiesterase enzymes in biological degradation of nucleic acids is known to those of skill in the art. Inhibition of the phosphodiesterase interferes with this degradation. The phosphodiesterase inhibitor according to this aspect of the invention may comprise, for example, caffeine and/or at least one methyl zanthene.

The epinephrine release stimulant stimulates the release of the hormone epinephrine, otherwise known as adrenaline. The inclusion of this stimulant increases epinephrine concentration in the bloodstream, and correspondingly provides the known effects of increased heart rate, vasoconstriction, elevated blood pressure and increased blood sugar levels. Examples of epinephrine release stimulants include caffeine and Ridlin.

In accordance with another aspect of the invention, a method is provided for enhancing mental acuity and physical energy in a subject, wherein the method comprises administering to the subject a composition comprising thiamine disulfide diisobutyrate and at least one of an adenosine antagonist, a phosphodiesterase, an epinephrine release stimulant, and a cyclic AMP enhancer. The method may comprise including any combination of the adenosine antagonist, a cyclic AMP enhancer, phosphodiesterase inhibitor, and epinephrine release stimulant, or all of them.

Additional advantages and modifications will readily occur to those skilled in the art. Therefore, the invention in its broader aspects is not limited to the specific details, representative devices and methods, and illustrative examples shown and described. Accordingly, departures may be made from such details without departing from the spirit or scope of the general inventive concept as defined by the appended claims and their equivalents.

What is claimed is:

1. A composition comprising thiamine disulfide diisobutyrate and caffeine.

2. A composition as recited in claim 1, wherein the caffeine is present in an amount of at least about 50 milligrams per daily dose.

3. A composition as recited in claim 1, wherein the caffeine is present in an amount of about 50 milligrams per daily dose to about 250 milligrams per daily dose.

4. A composition as recited in claim 1, wherein the caffeine is present in an amount of about 125 milligrams per daily dose.

5. A composition as recited in claim 1, wherein the caffeine is present in an amount of about 250 milligrams per daily dose.

6. A composition as recited in claim 1, wherein the thiamine disulfide diisobutyrate is present in an amount of at least about 200 milligrams per daily dose.

7. A composition as recited in claim 1, wherein the thiamine disulfide diisobutyrate is present in an amount of about 200 milligrams per daily dose to about 400 milligrams per daily dose.

8. A composition as recited in claim 1, wherein the thiamine disulfide diisobutyrate is present in an amount of about 200 milligrams per daily dose.

9. A composition as recited in claim 1, wherein the thiamine disulfide diisobutyrate is present in an amount of about 400 milligrams per daily dose.

10. A composition as recited in claim 1, further comprising an excipient.

11. A composition as recited in claim 1, further comprising a microemulsifying agent for microemulsifying the thiamine disulfide diisobutyrate and the caffeine.

12. A composition as recited in claim 1, wherein the composition comprises about 2.5 to 10 parts thiamine disulfide diisobutyrate for each part of the caffeine.

13. A composition as recited in claim 1, wherein the composition comprises about 2.5 parts thiamine disulfide diisobutyrate for each part of the caffeine.

14. A composition as recited in claim 1, wherein the composition comprises about 10 parts thiamine disulfide diisobutyrate for each part of the caffeine.

15. A composition as recited in claim 1, wherein the composition is a solid.

16. A composition as recited in claim 1, wherein the composition is located in a capsule.

17. A composition as recited in claim 1, wherein the composition is in tablet form.

18. A composition as recited in claim 1, wherein the composition is in a microemulsified form.

19. A composition comprising thiamine disulfide diisobutyrate and at least one of an adenosine antagonist, a cyclic AMP enhancer, a phosphodiesterase inhibitor, and an epinephrine release stimulant.

20. A composition as recited in claim 19, wherein the composition comprises thiamine disulfide diisobutyrate, the adenosine antagonist, the cyclic AMP enhancer, the phosphodiesterase inhibitor, and the epinephrine release stimulant.

21. A composition as recited in claim 20, wherein the composition comprises ephedrine.

22. A composition as recited in claim 19, comprising the thiamine disulfide diisobutyrate and the adenosine antagonist.

23. A composition as recited in claim 22, wherein the adenosine antagonist comprises caffeine.

24. A composition as recited in claim 19, comprising the thiamine disulfide diisobutyrate and the cyclic AMP enhancer.

25. A composition as recited in claim 24, wherein the cyclic AMP enhancer comprises at least one of forskohlin and sclareline.

26. A composition as recited in claim 19, comprising the thiamine disulfide diisobutyrate and the phosphodiesterase inhibitor.

27. A composition as recited in claim 26, wherein the phosphodiesterase inhibitor comprises caffeine.

28. A composition as recited in claim 26, wherein the phosphodiesterase inhibitor comprises at least one methyl zanthene.

29. A composition as recited in claim 19, comprising the thiamine disulfide diisobutyrate and the epinephrine release stimulant.

30. A composition as recited in claim 29, wherein the epinephrine release stimulant comprises caffeine.

31. A composition as recited in claim 30, wherein the epinephrine release stimulant comprises Ridlin.

32. A method for enhancing mental acuity and physical energy in a subject, the method comprising administering to the subject a composition comprising thiamine disulfide diisobutyrate and caffeine.

33. A method for enhancing mental acuity and physical energy in a subject, the method comprising administering to the subject a composition comprising thiamine disulfide diisobutyrate and at least one of an adenosine antagonist, a cyclic AMP enhancer, a phosphodiesterase, and an epinephrine release stimulant.

34. A method for making a composition comprising thiamine disulfide diisobutyrate, caffeine and an excipient, the method comprising:
(a) bringing the excipient to about 45° C.;
(b) after completing (a), mixing the thiamine disulfide diisobutyrate with the excipient at about 45° C. until a thiamine disulfide diisobutyrate solution is obtained;
(c) after completing (b), mixing the caffeine with the thiamine disulfide diisobutyrate solution at about 45° C. until a composition solution is obtained. 5.1 A method as recited in claim 5, further comprising:
bringing the composition solution to about 40° C.; and
filling a capsule with the composition solution at about 40° C.
